(19) United States
(12) Patent Application Publication
(10) Pub. No.: US 2014/0315282 A1
(43) Pub. Date: Oct. 23, 2014

(54) MICROINJECTOR MOUNTING MODULE AND NEEDLE-HOLDER COMPONENT

(71) Applicant: HUNAN CHANGSHA AO FIGURE BIOLOGICAL TECHNOLOGY CO., LTD., Changsha (CN)

(72) Inventor: Jianjun Peng, Changsha (CN)

(21) Appl. No.: 14/358,036

(22) PCT Filed: Jan. 10, 2013
(86) PCT No.: PCT/CN2013/070289

§ 371 Date: May 13, 2014

(30) Foreign Application Priority Data
Nov. 18, 2011 (CN) 201120458951.5

(51) Int. Cl. C12N 15/89

(52) U.S. Cl. CPC C12N 15/89
USPC 435/285.1

(57) ABSTRACT

A microinjector needle mounting module and a needle holder assembly, comprising a micro-injection needle (1), a needle seat (2) and a needle holder (3); the microinjector mounting module is formed by means of inserting the rear end of the microinjector (1) into a hole in the base (21) of the needle seat; the needle holder (3) is provided with an inner hole (32) passing through the barrel body; a conical socket (24) is provided inside the needle seat (2) and one end of the needle-holder (3) is provided with a conical connector (31), said conical connector (31) and conical socket (24) thus constituting a mutually-complementary insertion connection. In addition to a micro-injection needle being assembled with the needle seat to form a mounting module, other micro-operation needles such as holding pipettes or biopsy needles can also be assembled into a mounting module with said needle seat.

### FIELD OF THE UTILITY MODEL

The present utility model relates to a microneedle, particular to a microinjector needle mounting module and needle holder assembly for assisted reproductive technology, which is mainly used in human assisted reproductive technology for intracytoplasmic sperm injection and other micromanipulations, and belongs to the field of medical devices.

### BACKGROUND OF THE UTILITY MODEL

In the recent decades, the number of infertile couples is gradually increasing. So far, approximately 15% to 20% couples around the world are unable to reproduce normally. Among the infertile couples, approximately 50% are caused by male subfertility. In 1992, Palerme, et al. reported the first test-tube baby born by applying intracytoplasmic sperm injection (ICSI). Since then, ICSI has become one of the most important techniques for treating male infertility, etc.

Whether the ICSI technique can achieve a successful pregnancy is affected by a variety of factors. Such factors are roughly divided into two categories: one is non-technical factors, such as the quality of the ovum and sperm gametes per se, and the environment within the maternal body; the other is technical factors, including the micromanipulation tool and the technical level of the micromanipulation operator. The most invasive operation in the assisted reproductive technology is considered to be direct injection of the sperm into the cytoplasm of an oocyte through a microinjector needle. Therefore, the quality of the micromanipulation tool especially the microinjector needle, and the operational level of the micromanipulation operator are the most important factors which affect the success rate of ICSI. The technical level of a micromanipulation operator can continue to accumulate and improve in practice. However, the good level of a well-trained micromanipulation operator is often constrained by the micromanipulation tool. Therefore, the most critical factor that affects the success rate of ICSI on the technical level is the micromanipulation tool.

The micromanipulation tool comprises two parts: a microscope and a micromanipulation system. Currently, the microscopes manufactured by brand companies have excellent performance and reliable quality. When using such microscopes, it is generally required to perform only simple operations such as adjusting the focal length and transforming the magnification, which are less affected by the operator. However, the micromanipulation system requires complicated debugging and controlling by the operator to achieve its function, which are greatly affected by the operator and will in turn affect the operational level of the operator. The micromanipulation system comprises two parts: a positioning system and an injection system. The positioning system is used to control the up and down, back and forth, as well as left and right spatial positions of the controlled subject. The injection system comprises four parts: a microinjector, a connecting tube, a needle holder and a microinjector needle. The microinjector, the connecting tube and the needle holder are connected together through threaded joints, and the inner hole connected together is filled with paraffin oil. The microinjector needle is a glass capillary tube without any append structures, and comprises a needle tip, a needle body and a needle tail. The traditional material for the needle holder is a hollow stainless steel tube, which fixes one end of the injection needle and generally comprises a special-shaped nut, an oriented inner core and a rubber ring. The oriented inner core is thin on one end and thick on the other end. The thin end stretches out through the hole the special-shaped nut, and the rest portion retains within the hollow space of the special-shaped nut. The oriented inner core has a through inner hole, the diameter of which is just for the microinjector needle to pass through. The terminal of the metal needle holder connected to the special-shaped nut retracts into an inner cavity for accommodating the rubber ring. After the rear end of the microinjector needle passes through the oriented inner core, and then passes through the rubber ring to arrive at certain depth in the inner hole of the needle holder, the special-shaped nut is fastened, and the rubber ring is pressed through the oriented inner core, to reduce the inner diameter of the rubber ring hole and seal the gap between the rubber ring and the inner cavity of the needle holder terminal, so as to realize the purpose of fixing the injection needle. Through adjusting the pressure of the microinjector, the paraffin oil passes through the connecting tube and the needle holder, and flows into a suitable position of the needle body of the injection needle from the rear end of the microinjector needle, to complete the mounting of the microinjector needle.

An ideal mounting of the microinjector needle is required to meet the following requirements: when adjusting the pressure of the microinjector, the change of pressure can smoothly, faithfully (without suddenly from fast to slow, which is so-called “lag” phenomenon) transmit to the needle tip of the microinjector needle. However, in order to achieve the ideal effect by a traditional microinjection system, the micromanipulation operator is required to have rich experience and patience. It is full of great uncertainty, and a slightest mistake will produce the “lag” phenomenon. Such uncertainty is determined by the structure of the traditional needle holder, which mainly manifested in the following three aspects. Firstly, in the conventional operation, the microinjector needle will be pulled out and discarded after use. The negative pressure caused by the action of pulling out will cause the space in the terminal of the traditional needle holder (comprising the oriented inner core, the inner hole of the rubber ring and the inner hole of the needle holder part) to suck in air, which is mixed with the paraffin oil to form bubbles. Before mounting the next injection needle, these bubbles need to be discharged. The general operating step is to unscrew the special-shaped nut, to adjust the microinjector in the forward direction, so that the paraffin oil will flow out of the oriented inner core, until empirically no bubble is visible. When the bubbles are very small, they will adhere to the oriented inner hole, the rubber ring and the inner hole of the needle holder. Meanwhile, the operator will frequently consider the burdensome of filling paraffin into the oil line so as to discharge less paraffin oil, which will also cause that the bubbles cannot be eliminated completely. The bubbles are compressible and have severe “lag” effect on the transmission of pressure, which are the most important and most common reason leading to the “lag” phenomenon. Secondly, when mounting the microinjector needle, the rubber ring has to be passed through. Repeated passing through for many times will often scrape rubber off the rubber ring, forming rubber chips. These rubber chips will retain in the inner hole of the needle holder, the inner hole of the rubber ring, and the inner hole of the oriented inner core, causing an impeded oil line, and thus producing the “lag” phenomenon. Thirdly, when mounting and dismantling the microinjector needle, if the rear end of the microinjector needle breaks off and forms broken bodies of the glass capillary tube, they will retain in the inner hole of the oriented inner core. When the broken bodies are very short, they are difficult to be perceived by the operator. When mounting the next microinjector needle, the broken bodies of the glass capillary tube will be pushed into the inner hole of the needle holder, so as to block the oil line and produce the “lag” phenomenon.

In the traditional micromanipulation systems, there are various methods for removing bubbles, rubber chips and broken bodies of the glass capillary tube. For the bubbles, the general treating method is to unscrew the special-shaped nut, and adjust the microinjector in the forward direction, so that the paraffin oil will flow out of the oriented inner core, until empirically no bubble is visible. This method will consume more paraffin oil. However, if bubbles are not discovered by this method while the “lag” phenomenon indeed exists, more burdensome method will be required to further examine and observe whether there are bubbles, rubber chips and broken bodies of the glass capillary tube in the inner hole of the needle holder, the inner hole of the rubber ring, and the inner hole of the oriented inner core. The detecting method is as follows: unscrewing the special-shaped nut, dismantling the oriented inner core, picking out the rubber ring, and separating the needle holder with the connecting tube, then passing a steel needle into the oriented inner core, the rubber ring and the inner hole of the barrel body of the needle holder, and carefully observing whether bubbles, rubber chips and broken bodies of glass capillary tube are pounded out. The whole process is oil-consuming, and reassembling the dismantled components will further produce the risk of forming bubbles.

In summary, the traditional microinjector needle mounting systems have the following defects. Firstly, the structure is complicated, and there are design defects of producing bubbles, plastic chips, and broken bodies of glass capillary tube, etc. which lead to the “lag” phenomenon. Secondly, the method for removing the “lag” phenomenon is burdensome, and the result is not necessarily reliable. These defects have become important adverse factors affecting the success rate of ICSI.

Therefore, in order to solve the above-mentioned technical problems, it is indeed necessary to provide a microinjector needle mounting module and needle holder assembly having improved structures, to overcome the defects in the prior art.

### SUMMARY OF THE UTILITY MODEL

To solve the above-mentioned problems, the purpose of the present utility model is to provide a microinjector needle mounting module and needle holder assembly, which is simple in structure, easy in operation, stable in working state, and is able to overcome the defects present in traditional microinjector needle mounting systems.

To realize the above-mentioned purpose, the present utility model employs the following technical solution: a microinjector needle mounting module and needle holding assembly, comprising a microinjector needle, a needle seat, and a needle holder; wherein, the microinjector needle mounting module is assembled by inserting the rear end of the microinjector needle into a hole in the base of the needle seat; the needle holder is provided with a inner hole passing through the barrel body; a conical socket is provided within the needle seat, a conical connector is provided at one end of the needle holder, and the conical connector and the conical socket thus form a mutually-complementary insertion connection.

The microinjector needle mounting module and needle holder assembly of the present utility model is further set in such a way that the needle seat is a needle seat made of transparent or semitransparent material.

The microinjector needle mounting module and needle holder assembly of the present utility model is further set in such a way that the needle seat is a needle seat made of plastic or rubber material.

The microinjector needle mounting module and needle holder assembly of the present utility model is further set in such a way that the needle holder is a needle holder made of transparent or semitransparent material.

The microinjector needle mounting module and needle holder assembly of the present utility model is further set in such a way that the needle holder is a needle holder made of plastic or organic glass material.

The microinjector needle mounting module and needle holder assembly of the present utility model is further set in such a way that the needle seat comprises a base for fixing the microinjector needle and a mounting seat, an inner hole of the base for fixing the microinjector needle is provided in the base for fixing the microinjector needle, and the conical socket for connecting the needle holder is provided in the mounting seat.

The microinjector needle mounting module and needle holder assembly of the present utility model is further set in such a way that a restrictor ring is provided between the inner hole of the base and the conical socket, and the inner hole of the seat and the conical socket are in communication at the restrictor ring.

In comparison with the prior art, the present utility model has the following advantageous effects:

1. the needle seat and the needle holder are transparent or semitransparent, so that visualization can be realized, and it is more visually to detect bubbles and various other abnormalities;

2. the needle holder is simple in structure and has no additional accessories, so as to avoid the “lag” problem caused by rubber chips and broken bodies of glass capillary tube;

3. the microinjector needle mounting module and the needle holder are closely connected through the a mutually-complementary insertion connection formed by the conical socket and the conical connector, and are convenient and efficient to amount and dismantle;

4. there is no need to discharging paraffin oil when mounting the microinjector needle, so that the consuming amount of paraffin oil is reduced, and the number of people for the micromanipulation after each time of paraffin oil filling is increased.

### DESCRIPTION OF THE DRAWINGS

FIG. 1 is the schematic diagram of the needle seat according to the microinjector needle mounting module and needle holder assembly of the present utility model.

FIG. 2 is the schematic diagram of the microinjector needle mounting module according to the microinjector needle mounting module and needle holder assembly of the present utility model.

FIG. 3 is the schematic diagram of the needle holder according to the microinjector needle mounting module and needle holder assembly of the present utility model.

FIG. 4 is the schematic diagram of the connection between the microinjector needle mounting module and the needle holder according to the microinjector needle mounting module and needle holder assembly of the present utility model.

### DETAILED EMBODIMENTS

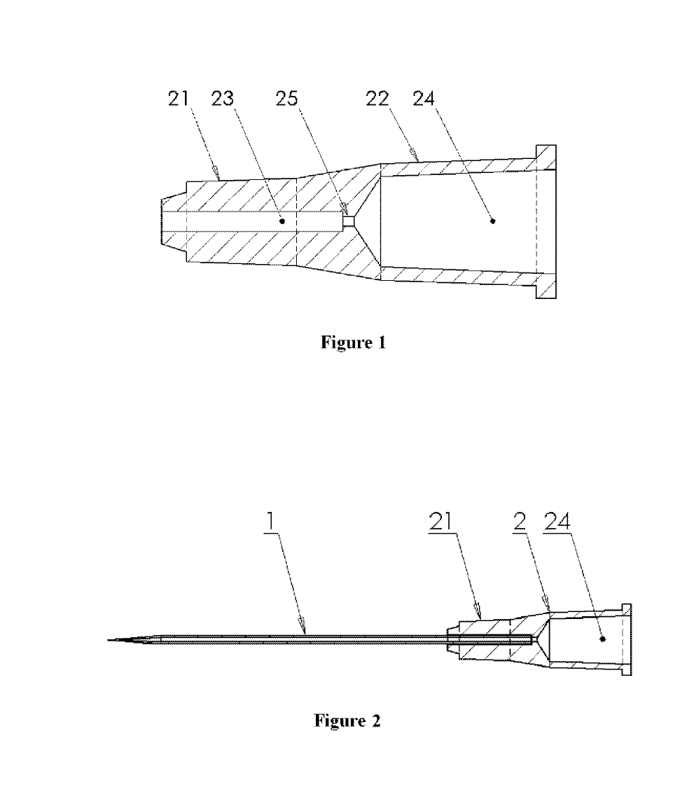

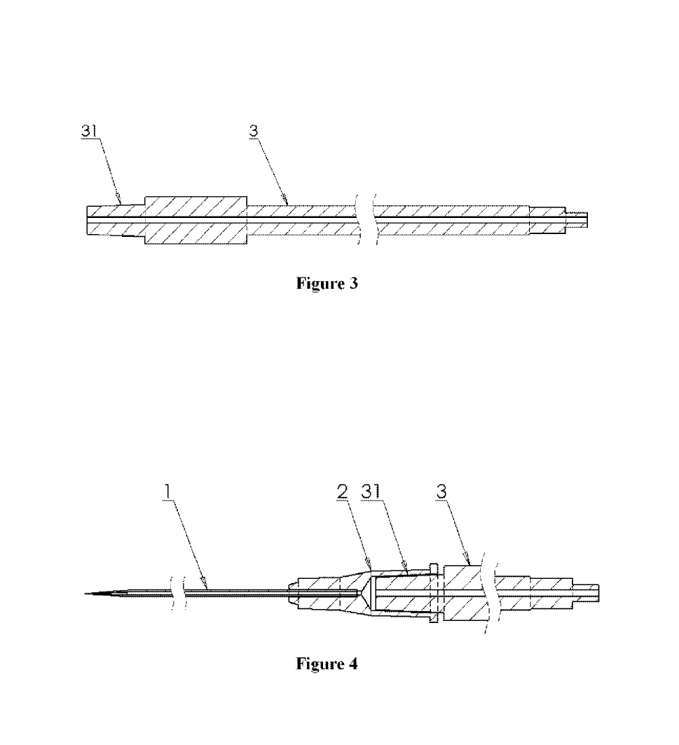

Please refer to FIG. 1, FIG. 2 and FIG. 3 of the present specification. The present utility model is a microinjector needle mounting module and needle holder assembly, which consists of three parts of microinjector needle 1, needle seat 2, and needle holder 3. Wherein, the rear end of the microinjector needle 1 is inserted into the needle seat 2 to assemble into the microinjector needle mounting module.

The needle seat 2 is made of transparent or semitransparent plastic or rubber material having good elasticity. Particularly, the needle seat 2 comprises a base 21 for fixing microinjection and a mounting seat 22. An inner hole of the base 23 for fixing the microinjector needle 1 is provided within the base 21 for fixing the microinjector needle, and the conical socket 24 is provided on the mounting seat 22. A restrictor ring 25 is provided between the inner hole of the base 23 and the conical socket 24. The inner hole of the base 23 and the conical socket 24 are in communication at the restrictor ring 25.

The needle holder 3 is also made of transparent or semitransparent plastic or organic glass material having good rigidity. Particularly, the needle holder is provided with a inner hole 32 passing through the barrel body, which is provided with a conical connector 31 at one end. The conical connector 31 and the conical socket 24 form a mutually-complementary insertion connection, so as to mount the microinjector needle mounting module onto the needle holder 3.

It needs to be noted that, in addition to the microinjector needle 1, other micromanipulation needles such as holding pipettes and biopsy needles can also be assembled into a mounting module with the needle seat 2.

The detailed embodiments above are only preferred examples of the present creation, and are not to limit the present creation. All the modifications, equivalent substitutions, improvements, etc. made within the spirit and principle of the present creation should be included within the protection scope of the present creation.

1. A microinjector needle mounting module and needle holder assembly, characterized in that it comprises a microinjector needle, a needle seat and a needle holder; wherein, the microinjector needle mounting module is assembled by inserting the rear end of the microinjector needle into a hole in the base of the needle seat; the needle holder is provided with a inner hole passing through the barrel body; a conical socket is provided within the needle seat, a conical connector is provided at one end of the needle holder, the conical connector and the conical socket thus form a mutually-complementary insertion connection.

2. The microinjector needle mounting module and needle holder assembly according to claim 1, characterized in that the needle seat is a needle seat made of transparent or semitransparent material.

3. The microinjector needle mounting module and needle holder assembly according to claim 1, characterized in that the needle seat is a needle seat made of plastic or rubber material.

4. The microinjector needle mounting module and needle holder assembly according to claim 1, characterized in that the needle holder is a needle holder made of transparent or semitransparent material.

5. The microinjector needle mounting module and needle holder assembly according to claim 1, characterized in that the needle holder is a needle holder made of plastic or organic glass material.

6. The microinjector needle mounting module and needle holder assembly according to claim 1, characterized in that the needle seat comprises a base for fixing microinjector needle and a mounting seat, an inner hole of the base for fixing the microinjector needle is provided within the base for fixing the microinjector needle, and the conical socket for connecting the needle holder is provided on the mounting seat.

7. The microinjector needle mounting module and needle holder assembly according to claim 6, characterized in that a restrictor ring is provided between the inner hole of the base and the conical socket, and the inner hole of the base and the conical socket are in communication at the restrictor ring.
